(19) United States
(12) Patent Application Publication
(10) Pub. No.: US 2010/0122362 A1
(43) Pub. Date: May 13, 2010

(54) CELLS EXPRESSING ALPHA-SYNUCLEIN AND USES THEREFOR

(75) Inventor: Feng Liang, Sudbury, MA (US)

Correspondence Address: FISH & RICHARDSON P.C., P.O. BOX 1022, MINNEAPOLIS, MN, 55440-1022, US

(73) Assignee: FoldRx Pharmaceuticals, Inc., Cambridge, MA (US)

(21) Appl. No.: 12/445,012

(22) PCT Filed: Oct. 12, 2007
(86) PCT No.: PCT/US07/81227

§ 371 Date: Jan. 12, 2010

Related U.S. Application Data
(60) Provisional application No. 60/829,302, filed on Oct. 13, 2006.

(51) Int. Cl. A01K 67/027; C12N 5/0793; G01N 33/53; A61K 31/352; A61K 31/497; A61P 25/28

(52) U.S. Cl. 800/14; 435/325; 435/7.21; 514/455; 514/256

(57) ABSTRACT

Disclosed are mammalian neuronal cells expressing alpha synuclein as well as methods of screening to identify compounds that decrease toxicity induced by alpha synuclein expression. Compounds identified by such screens can be used to treat or prevent synucleinopathies such as Parkinson's disease, familial Parkinson's disease, Lewy body disease, the Lewy body variant of Alzheimer's disease, dementia with Lewy bodies, multiple system atrophy, or the Parkinsonism-dementia complex of Guam.

### CROSS-REFERENCE TO RELATED APPLICATIONS

This application claims priority from U.S. Provisional Application No. 60/829,320, filed Oct. 13, 2006. The entire content of the prior application is incorporated herein by reference.

### TECHNICAL FIELD

The invention relates to cells expressing alpha-synuclein and methods of screening to identify compounds that decrease toxicity induced by alpha-synuclein expression.

### BACKGROUND

Parkinson's disease is a neurodegenerative disorder that is pathologically characterized by the presence of intracytoplasmic Lewy bodies (Lewy in Handbuch der Neurologie, M. Lewandowski, ed., Springer, Berlin, pp. 920-933, 1912; Pollanen et al.,J. Neuropath. Exp. Neurol. 52:183-191, 1993), the major components of which are filaments consisting of the 140-amino acid alpha-synuclein protein (Spillantini et al., Proc. Natl. Acad. Sci. USA 95:6469-6473, 1998; Arai et al., Neurosci. Lett. 259:83-86, 1999; Ueda et al., Proc. Natl. Acad. Sci. USA 90:11282-11286, 1993). Three dominant mutations in alpha-synuclein causing familial early onset Parkinson's disease have been described, suggesting that Lewy bodies contribute mechanistically to the degeneration of neurons in Parkinson's disease and related disorders (Polymeropoulos et al., Science 276:2045-2047, 1997; Kruger et al., Nature Genet. 18:106-108, 1998; Zarranz et al., Ann. Neurol. 55:164-173, 2004). Triplication and duplication mutation of the alpha-synuclein gene have been linked to early-onset of Parkinson's disease (Singleton et al., Science 302:841, 2003; Chartier-Harlin at al. Lancet 364:1167-1169, 2004; Ibanez et al., Lancet 364:1169-1171, 2004).

The common involvement of alpha-synuclein in a spectrum of diseases such as Parkinson's disease, dementia with Lewy bodies, multiple system atrophy and the Lewy body variant of Alzheimer's disease has led to the classification of these diseases under the umbrella term of “synucleinopathies.” Protecting neurons from the toxic effects of alpha-synuclein is a promising strategy for treating these diseases. There is thus a need for systems that permit the identification of compounds and compositions that prevent or suppress alpha-synuclein induced toxicity in neuronal cells. Such compounds and compositions are useful in treating or preventing synucleinopathies.

### SUMMARY

The invention is based, at least in part, on the discovery that alpha-synuclein can be stably expressed in a mammalian cell of neuronal origin via an inducible expression system such that the alpha-synuclein expression alone is toxic to the cells without the need for co-treatment of the cells with an additional composition (e.g., a toxicity-inducing agent or a neuronal differentiation factor).

The present invention features a mammalian neuronal cell containing a stably integrated expression construct containing an inducible promoter operably linked to a nucleic acid encoding a protein comprising a human alpha synuclein, where induction of expression of the nucleic acid, in the absence of co-treatment of the cell with a toxicity-inducing agent or a neuronal differentiation factor, results in a decrease in cell viability. The alpha-synuclein can be, for example, wild type alpha-synuclein or a mutant alpha-synuclein (e.g., the A53T mutant human alpha-synuclein, the A30P mutant human alpha-synuclein, or the E46K mutant human alpha-synuclein). The alpha synuclein can be a full-length alpha-synuclein or a functional fragment of alpha synuclein. The mammalian neuronal cell can be a human neuronal cell. The mammalian neuronal cell can also be, or can be derived from, a neuronal cell line (e.g., an H4 cell line, a PC12 cell line, a SK-N-SH cell line, a SH-SY5Y cell line, a Neuro-2a cell line, a U87 MG cell line, or any other neuronal cell line described herein). The protein can be a fusion protein containing a detectable protein, e.g., a fluorescent protein, an enzyme, or an epitope. In some embodiments, the fluorescent protein can be red fluorescent protein, green fluorescent protein, blue fluorescent protein, yellow fluorescent protein, and cyan fluorescent protein. In some embodiments, the enzyme can be beta-galactosidase, luciferase, alkaline-phosphatase, or horseradish peroxidase. In some embodiments, the epitope can be a FLAG, HA, His6, AU1, Tap, Protein A, or MYC epitope. The mammalian neuronal cell can be an isolated cell or can be in or on a non-human mammal (e.g., a mouse, a rat, a rabbit, a guinea pig, a goat, a dog, a cat, a horse, a cow, a whale, or a monkey).

In some embodiments, the mammalian neuronal cell can also contain a stably integrated repressor construct that constitutively expresses a repressor protein, where (i) in the absence of an exogenously added inducer, the repressor protein binds to the inducible promoter and suppresses expression of the nucleic acid, and (ii) in the presence of the exogenously added inducer, the repressor protein binds to the inducer and the nucleic acid is expressed. The repressor can be a Tet Repressor, e.g., a reverse tetracycline-controlled transactivator (rtTA). The inducer can be tetracycline or an derivative or analogue of tetracycline such as doxycycline.

Also featured herein is a non-human mammal comprising any of the mammalian neuronal cells described above. The non-human mammal can be any of the non-human mammals described herein.

The invention also provides a method of inducing toxicity in a mammalian neuronal cell, which includes the step of inducing a level of expression of the nucleic acid (e.g., a nucleic acid containing a coding sequence for an alpha-synuclein) in a mammalian neuronal cell that is toxic to the cell. The mammalian neuronal cell can be any of those described above. Thus, the alpha-spuclein can be any of those described herein.

Also provided is a method of inducing toxicity in a mammalian neuronal cell. The method includes the steps of contacting any of the neuronal cells described above with an amount of an exogenously added inducer effective to induce expression of the nucleic acid (e.g., a nucleic acid containing a coding sequence for an alpha-synuclein) and toxicity in the cell. The alpha-synuclein can be any alpha-synuclein described herein.

Also featured herein is a method of identifying a compound that prevents or suppresses alpha-synuclein-induced toxicity, which includes the steps of: (i) culturing any of the mammalian neuronal cells described above in the presence of a candidate agent and under conditions that allow for expression of the nucleic acid at a level that, in the absence of the candidate agent, is sufficient to induce toxicity in the cell; (ii) measuring cell viability in the presence of the candidate agent; and (iii) comparing cell viability measured in the presence of the candidate agent to cell viability in the absence of the candidate agent, where if cell viability is greater in the presence of the candidate agent as compared to in the absence of the candidate agent, then the candidate agent is identified as a compound that prevents or suppresses alpha-synuclein-induced toxicity.

Also provided is a method of identifying a compound that prevents or suppresses alpha-synuclein-induced Golgi fragmentation, which includes the steps of: (i) culturing any of the mammalian neuronal cells described above in the presence of a candidate agent and an amount of the exogenously added inducer effective to induce expression of the nucleic acid at a level that, in the absence of the candidate agent, induces Golgi-fragmentation in the cell; (ii) measuring Golgi fragmentation in the cell in the presence of the candidate agent; and (iii) comparing Golgi fragmentation in the cell measured in the presence of the candidate agent to Golgi fragmentation in the absence of the candidate agent, where if Golgi fragmentation is less in the presence of the candidate agent as compared to in the absence of the candidate agent, then the candidate agent is identified as a compound that prevents or suppresses alpha-synuclein-induced Golgi fragmentation.

Also featured is a method of identifying a compound that increases endoplasmic reticulum to Golgi vesicular trafficking. The method includes the steps of: (i) culturing the cell of any of claims 15-17 in the presence of a candidate agent and an amount of the exogenously added inducer effective to induce expression of the nucleic acid at a level that, in the absence of the candidate agent, reduces endoplasmic reticulum to Golgi vesicular trafficking in the cell; (ii) measuring endoplasmic reticulum to Golgi vesicular trafficking in the cell in the presence of the candidate agent; and (iii) comparing endoplasmic reticulum to Golgi vesicular trafficking in the cell measured in the presence of the candidate agent to endoplasmic reticulum to Golgi vesicular trafficking measured in the absence of the candidate agent, where an increase in endoplasmic reticulum to Golgi vesicular trafficking in the cell in the presence of the candidate agent as compared to endoplasmic reticulum to Golgi vesicular trafficking in the absence of the candidate agent identifies the candidate agent as a compound that increases endoplasmic reticulum to Golgi vesicular trafficking.

Also featured is a method of identifying a compound that increases vesicle docking and fusion, which includes the steps of: (i) culturing any of the mammalian neuronal cells described above in the presence of a candidate agent and an amount of the exogenously added inducer effective to induce expression of the nucleic acid at a level that, in the absence of the candidate agent, reduces vesicle docking and fusion in the cell; (ii) measuring vesicle docking and fusion in the cell in the presence of the candidate agent; and (iii) comparing vesicle docking and fusion in the cell in the presence of the candidate agent to vesicle docking and fusion in the absence of the candidate agent, where an increase in vesicle docking and fusion in the presence of the candidate agent as compared to vesicle docking and fusion in the absence of the candidate agent identifies the candidate agent as a compound that increases vesicle docking and fusion.

The invention also provides a method of identifying a compound that increases the secretion of a protein from a cell. The method includes the steps of (i) culturing any of the mammalian neuronal cells described above in the presence of a candidate agent and an amount of the exogenously added inducer effective to induce expression of the nucleic acid at a level that, in the absence of the candidate agent, reduces protein secretion from the cell; (ii) measuring protein secretion from the cell in the presence of the candidate agent; and (iii) comparing protein secretion from the cell in the presence of the candidate agent to protein secretion in the absence of the candidate agent, where an increase in protein secretion from the cell in the presence of the candidate agent as compared to protein secretion in the absence of the candidate agent identifies the candidate agent as a compound that increases protein secretion from the cell.

Also featured is a method of treating a synucleinopathy, which includes the steps of: administering to an individual (e.g., a human patient) diagnosed as having a synucleinopathy a pharmaceutical composition comprising a therapeutically effective amount of a compound identified by any of the methods described herein. Optionally, the method can also include the step of selecting and/or diagnosing the individual as one having, or at risk of developing, a synucleinopathy. The synucleinopathy can be, for example, Parkinson's disease, familial Parkinson's disease, Lewy body disease, the Lewy body variant of Alzheimer's disease, dementia with Lewy bodies, multiple system atrophy, or the Parkinsonism-dementia complex of Guam. The synucleinopathy can be one caused by a mutation in alpha-synuclein such as any of those described herein.

A “toxicity-inducing agent” is a composition that induces a level of toxicity in a cell expressing alpha synuclein that exceeds the level of toxicity (if any) resulting from either expressing alpha synuclein alone in the cell or contacting the cell with the toxicity-inducing agent alone. A “toxicity-inducing agent” can be a compound (e.g., a proteasome inhibitor) that is administered at levels non-toxic to a cell on its own but triggers toxicity when combined with expression of alpha synuclein. In some embodiments, the “toxicity-inducing agent” can be one or more genetic elements (e.g., mutations such as inactivating mutations in a gene) that trigger or otherwise sensitize a mammalian cell for cytotoxicity when combined with alpha-synuclein expression. For example, the mutation could be the ts41 mutation of APPBP 1, which results in defects of the ubiquitin-proteasome pathway.

As used herein, a “neuronal differentiation factor” is a protein that triggers differentiation of a neuronal cell. Examples of neuronal differentiation factors include NGF, BDNF, Neurotrophin-3, and Neurotrophin-4.

An advantage of the neuronal cells described herein is that they permit the stable, regulated expression of alpha-synuclein (including wild type alpha-synuclein) such that alpha-synuclein expression alone is toxic to the cells. As such, compounds identified as rescuing the toxicity that occurs in these alpha-synuclein expressing cells are expected to mediate their beneficial effect via an alpha-synuclein related pathway rather than by acting on, for example, a toxicity-inducing agent or some other co-factor used to induce cytotoxicity.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, the exemplary methods and materials are described below. All publications, patent applications, patents, and other references mentioned herein are incorporated by reference in their entirety. In case of conflict, the present application, including definitions, will control. The materials, methods, and examples are illustrative only and not intended to be limiting.

Other features and advantages of the invention will be apparent from the following detailed description, and from the claims.

### BRIEF DESCRIPTION OF THE DRAWINGS

FIG. 1 is a schematic diagram depicting the tetracycline-regulated, conditional expression of wild type alpha-synuclein in H4 cells. Tetracycline-regulated expression of alpha-synuclein was through the use of regulatory elements from the E. coli Tn10-encoded tetracycline (Tet) resistance operon. Two expression cassettes, expressing the Tet repressor (TR) or alpha-synuclein (Syn), were integrated into host cell genome via lentiviral systems. The Tet-repressor is constitutively transcribed under the CMV promoter. Two tetracycline-responsible-elements (TREs) were inserted in the promoter region. The Tet-repressor binds to the TRE and alpha-synuclein transcription is silenced. In the presence of tetracycline (i.e., the induced condition), the binding of tetracycline to the Tet-repressor releases the protein from TRE, and results in the expression of alpha-synuclein.

FIG. 2 is a photograph of an immunoblot depicting the conditional overexpression of wild type alpha-synuclein in TS217 cells. TS217 cell lysates were collected before (−) or after induction with tetracycline for one day (1d) or three days (3d). Parental H4 (C) cell lysate was collected to indicate endogenous level of alpha-synuclein protein. Lysates were resolved by sodium dodecyl-sulfate polyacryamide gel electrophoresis (SDS-PAGE) and proteins were detected using antibodies specific for alpha-synuclein or actin where indicated at the left.

FIG. 3 is a line graph depicting progressive cytotoxicity in H4 cells overexpressing wild type alpha-synuclein. Alpha-synuclein was induced by the addition of tetracycline for 3-6 days. Cell viability was analyzed via the measurement of cellular ATP level. Relative cell viability was calculated as the ratio of induced cells to control cells, as an indication of alpha-synuclein -induced cytotoxicity. The Y-axis represents Relative Viability and the X-axis the number of days. Error bars represent the standard deviation of eight replicates.

FIG. 4A is a photograph of live TS217 cells. TS217 H4 cells were plated on glass tissue culture chamber slides coated with poly-L-lysine. The cells were further cultured for five days without (Control) or with tetracycline induction (Induced) of alpha-synuclein. Calcein AM staining of live cells was imaged via a fluorescent microscope (Olympus) with a digital camera controlled by the Metamorphprogram (Universal Imaging, West Chester, Pa.).

FIG. 4B is a bar graph depicting the quantitation of cytotoxicity determined by the calcein imaging of FIG. 4A, and quantitated using Metamorph software. Unpaired t test, ***P<0.0001.

FIG. 5 is a line sigmoidal curve-fit depicting the concentration-response of tetracycline in a cell viability assay. Stable H4 clone was incubated with different concentrations of tetracycline for six days in a 96-well plate. Cells were lysed and cellular ATP was measured. Media with vehicle alone served as control. Error bars represent the standard deviation of eight replicates. The X-axis represents the log of the concentration of tetracycline (in μg/mL) and the Y-axis represents the ATP concentration expressed as relative light units (RLU).

FIG. 6A is a photograph of cells and depicts fragmentation of the Golgi apparatus in wild type alpha-synuclein -overexpressed cells. TS217 H4 cells were plated on glass tissue culture chamber slides coated with poly-L-lysine. The cells were further cultured for six days without (Control) or with tetracycline induction (Induced) of alpha-synuclein. GM130-immunoreactive Golgi complex was imaged.

FIG. 6B is a bar graph depicting a quantitative analysis of cells (from FIG. 6A) with normal Golgi complex. Intact Golgi structure was captured by the exclusion of small foci representing fragmented Golgi. Unpaired t test, ***P<0.0001.

FIG. 7 is photograph of cultured cells depicting a double-immunofluorescence staining. H4 cells were plated on glass tissue culture chamber slides coated with poly-L-lysine. The cells were further cultured for six days without (Control) or with tetracycline induction (Induced) of alpha-synuclein. Cells were either stained using antibodies specific for GM130 or mannosidase II, respectively. A high magnification view of the Golgi apparatus clearly showed alterations in Golgi morphology in alpha-synuclein-overexpressed cells.

FIG. 8 is a photograph of cultured cells stained with mannosidase II or calnexin and depicting the integrity of the endoplasmic reticulum (ER) in tetracycline treated cells.

TS217 H4 cells were plated on glass tissue culture chamber slides coated with poly-L-lysine. The cells were further cultured for five days without (Control) or with tetracycline induction (Induced) of alpha-synuclein. Calnexin-immunoreactive ER and mannosidase II-immunoreactive Golgi complex were analyzed.

FIG. 9 is a bar graph depicting the rescue of alpha-synuclein-induced toxicity by a compound. TS217 cells were treated with tetracycline to induce alpha-synuclein expression for 5 days. During the 5 day induction, cells were also treated with various concentrations of 1-t-Butyl-3-(4-chloro-phenyl)-1H-pyrazolo[3,4-d]pyrimidin-4-ylamine (Cmp J) (0, 0.08 μM, 0.15 μM, and 0.3 μM). Relative viability was determined by measuring the ATP concentration in each cell set and was a ratio of induced to control as above. The Y-axis represents the Relative viability and the X-axis represents the concentration of compound. Asterisks indicate results of a Kruskal-Wallis test: *, **, ***: P<0.05, 0.01, and 0.001 respectively.

FIG. 10 is a bar graph depicting the effect of plating density on alpha-synuclein-induced cytotoxicity. Serially diluted TS217 cells were plated in 96 well plates at dilutions of 250, 500, 1000, 2000, and 4000 cells/well. Cells were incubated in the presence of tetracycline for five days. After incubation, cells were harvested and lysed to determine the intracellular ATP concentration. Relative cell viability was calculated as the ratio of induced cells to control cells. Error bars represent the standard deviation of six replicates.

FIG. 11 is a scatter plot depicting the inhibition of alpha-synuclein-induced cytotoxicity in TS217 cells by forskolin (FSK). The X-axis represents the dosage of FSK in micromolar (μM). The Y-axis represents the relative concentration of ATP (expressed as relative light units (RLUs)) in the TS217 cells as a function of cell-viability.

### DETAILED DESCRIPTION

### Alpha-Synuclein Nucleic Acids and Proteins

The compositions and methods disclosed herein use a protein comprising an alpha-synuclein polypeptide. The term “alpha synuclein” encompasses naturally occurring alpha synuclein sequences (e.g., naturally occurring human wild type and mutant alpha synucleins) as well as functional variants thereof.

Human alpha synuclein is encoded by the following nucleotide sequence:

(SEQ ID NO: 1)

atggatgtattcatgaaaggactttcaaaggccaaggagggagttgtggc
tgctgctgagaaaaccaaacagggtgtggcagaagcagcaggaaagacaa
aagagggtgttctctatgtaggctccaaaaccaaggagggagtggtgcat
ggtgtggcaacagtggctgagaagaccaaagagcaagtgacaatgttgga
ggagcagtggtgacgggtgtgacagcagtagcccagaagacagtggaggg
agcagggagcattgcagcagccactggctttgtcaaaaaggaccagttgg
gcaagaatgaagaaggagccccacaggaaggaattctggaagatatgcct
gtggatcctgacaatgaggcttatgaaatgccttctgaggaagggtatca
agactacgaacctgaagcctaa.

Human alpha synuclein has the following amino acid sequence:

(SEQ ID NO: 2)

MDVFMKGLSKAKEGVVAAAEKTKQGVAEAAGKTKEGVLYVGSKTKEGVVH
GVATVAEKTKEQVTNVGGAVVTGVTAVAQKTVEGAGSIAAATGFVKKDQL
GKNEEGAPQEGILEDMPVDPDNEAYEMPSEEGYQDYEPEA.

The term “variants” is used herein to include functional fragments, mutants and derivatives. For example, “variants” may include substitutions of naturally occurring amino acids at specific sites (e.g., conservative amino acid substitutions), including but not limited to, naturally and non-naturally occurring amino acids. In some embodiments, an alpha synuclein protein is at least 80%, 85%, 90%, 95%, or 98% identical to the amino acid sequence of SEQ ID NO:2 and retains alpha-synuclein function.

As used herein, “activity” or “function” of an alpha-synuclein includes, but is not limited to, an ability to cause cytotoxicity (e.g., loss of Golgi integrity and/or or induction of cell death (measured by, e.g., reduction in cellular ATP concentration or apoptosis)) when overexpressed in mammalian cell. While not limited by any particular mechanism of action, the cytotoxicity can be caused by formation of inclusions or aggregates in a cell or impaired proteasomal activity.

In some embodiments, a full-length alpha-synuclein protein can be used. The term “full-length” refers to an alpha-synuclein protein that contains all the amino acids encoded by the alpha-synuclein cDNA. In other embodiments, different lengths of the alpha-synuclein protein may be used. For example, only functionally active domains of the protein can be used. Thus, a protein fragment of almost any length can be employed, provided it is functional.

In certain embodiments, variants of the alpha-synuclein protein can be used. Such variants may include biologically-active fragments of the alpha-synuclein protein. These include proteins with alpha-synuclein activity that have amino acid substitutions. Alpha-synuclein mutants that can be expressed in mammalian cells include the A53T mutant (containing a substitution of an alanine for a threonine at position 53) and the A30P mutant (containing a substitution of an alanine for a proline at position 30).

In certain embodiments, fusion proteins including at least a portion of the alpha-synuclein protein may be used. For example, a portion of the alpha-synuclein protein may be fused with a second domain. The second domain of the fusion protein can be selected from the group consisting of: an immunoglobulin element (e.g., an Fc fragment of an immunoglobulin molecule), a dimerizing domain, a targeting domain, a stabilizing domain, and a purification domain. Alternatively, a portion of alpha-synuclein protein can be fused with a heterologous molecule such as a detection protein. Exemplary detection proteins include: (1) a fluorescent protein such as green fluorescent protein (GFP), cyan fluorescent protein (CFP) or yellow fluorescent protein (YFP); (2) an enzyme such as β-galactosidase or alkaline phosphatase (AP); and (3) an epitope such as glutathione-S-transferase (GST) or hemagluttin (HA). To illustrate, an alpha synuclein protein can be fused to GFP at the N- or C-terminus or other parts of the alpha-synuclein protein. These fusion proteins provide methods for rapid and easy detection and identification of the alpha-synuclein protein in the recombinant host cell, exemplified herein by the mammalian cell (e.g., a mammalian neuronal cell).

Also described herein are methods of preparing and transferring nucleic acids encoding an alpha-synuclein protein into a mammalian cell so that the cell expresses the alpha-synuclein protein. The term “alpha synuclein nucleic acid” encompasses a nucleic acid comprising a sequence as represented in SEQ ID NO: 1 as well as a nucleic acid encoding any of the variants of alpha-synuclein described herein. Exemplary alpha-synuclein nucleic acids include those encoding wild type human alpha-synuclein or the A53T or A30P mutant proteins.

The term “nucleic acid” generally refers to at least one molecule or strand of DNA, RNA or a derivative or mimic thereof, comprising at least one nucleobase, for example, a naturally occurring purine or pyrimidine base found in DNA or RNA. Generally, the term “nucleic acid” refers to at least one single-stranded molecule, but in specific embodiments will also encompass at least one additional strand that is partially, substantially or fully complementary to the at least one single-stranded molecule. Thus, a nucleic acid may encompass at least one double-stranded molecule or at least one triple-stranded molecule that comprises one or more complementary strand(s) or “complement(s)” of a particular sequence comprising a strand of the molecule.

### Nucleic Acid Vectors for Inducible Expression of Alpha-Synuclein in Mammalian Cells

An alpha synuclein nucleic acid can be transfected into a mammalian cell using nucleic acid vectors that include, but are not limited to, plasmids, linear nucleic acid molecules, artificial chromosomes, and viral vectors. The vectors can contain as a transgene any of the alpha-synuclein nucleic acids, mutant or variant forms of alpha-synuclein described herein.

Once the vector or nucleic acid molecule containing the construct(s) has been prepared for expression, the DNA construct(s) can be introduced into an appropriate host cell by any of a variety of suitable means, i.e., transformation, transfection, conjugation, protoplast fusion, electroporation, particle gun technology, calcium phosphate-precipitation, direct microinjection, and the like. Where the vector is a viral vector, the vector can be delivered to the cell by direct infection. Preferably the vector is stably integrated into the host genome. Methods of producing a cell line containing a stably integrated nucleic acid (e.g., a vector) are well known in the art (see, for example, Sambrook et al. in Molecular Cloning: A Laboratory Manual. Cold Spring Harbor Laboratory Press, NY, Vol. 1, 2, 3 (1989)). Briefly, a cell transfected with a nucleic acid can be selected for using a host of antibiotics including, for example, G418, neomycin, or hygromycin B. Generally, the transfected nucleic acid contains an suitable antibiotic resistance gene or is co-transfected with a vector containing a suitable antibiotic resistance gene. Thus, only cells and their progeny which contain a stably integrated nucleic acid encoding the antibiotic resistance gene will survive when grown in antibiotic.

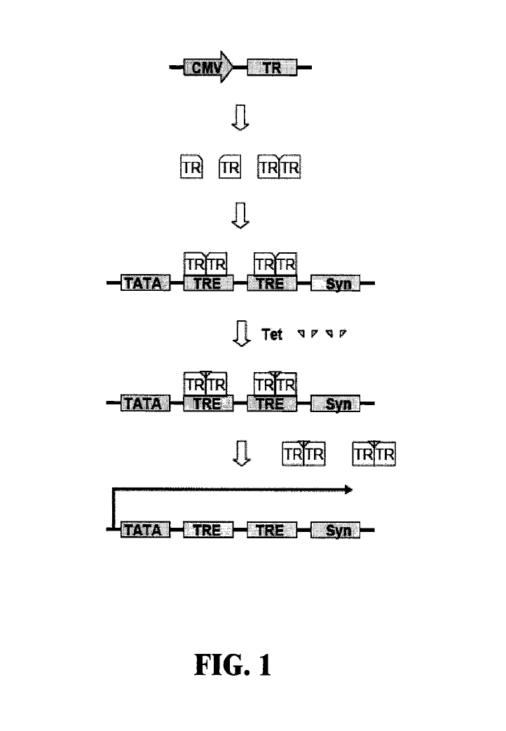

Alpha-synuclein can be expressed under the control of an inducible promoter. In general, inducible expression vectors are composed of one or more copies of an Inducer Responsive Element, which separates (i) a mammalian transcriptional promoter (e.g., the CMV promoter containing, e.g., the TATA box promoter element—the DNA docking site for the transcriptional machinery) and (ii) an operably linked nucleic acid encoding a transgene (e.g., an alpha-synuclein) of interest. Also present in the cell containing the expression vector is a transcriptional repressor protein which is capable of binding to the inducer responsive element in the absence of the inducer. The repressor can be endogenously expressed or be exogenously expressed in trans from a nucleic acid introduced to the cell. In addition to DNA binding, the transcriptional repressor can also bind to the inducer, wherein binding of the inducer to the repressor causes the repressor to dissociate from DNA (see FIG. 1). Thus, in the absence of an inducer (e.g., a compound such as doxycycline, tetracycline, or tamoxifen), the repressor protein binds to the one or more inducer responsive elements and prevents transcription of the transgene. However, upon binding to the inducer, the repressor protein disengages from the inducer responsive element and allows for the transcription and subsequent expression of the transgene. Generally, expression of the transgene following treatment with an inducer is dependent on the dosage of the inducer (e.g., the higher the concentration of inducer administered, the higher the transgene expression). Examples of such inducible expression vectors include, but are not limited to, the Tet-On vector systems such as those described below and ecdysone-inducible expression vector systems such as those produced by Stratagene Inc. (La Jolla, Calif.).

Alternatively, the cell containing the expression vector can contain a transcriptional activator capable of binding to the inducer responsive element(s) only in the presence of the inducer. Thus, administration of the inducer to the cells also “turns-on” expression of a transgene by inducing the binding of a positive transcription factor to the inducer responsive element. Example of such vectors and regulatory systems include the estrogen/tamoxifen-regulated estrogen receptor vectors systems.

In some embodiments, an alpha-synuclein polypeptide can be expressed under control of a “Tet-On” system as is exemplified in the Examples below (also see FIG. 1). The Tet-on vector contains a tetracycline-responsive element (TRE), which is bound by the tetracycline-controlled trans-repressor, rtTA. A variant form of a wild-type tet-repressor (TetR), rtTA is a fusion polypeptide composed of the TetR repressor and the VP16 transactivation domain of Herpes Symplex virus type-1. A four amino acid change in the wild-type TetR DNA binding domain alters the rtTA DNA binding characteristics such that it can only recognize the TetO sequences in the tetracycline response element (TRE) of the target transgene in the presence of the tetracycline or doxycycline effector. Thus, in the Tet-On system, transcription of the TRE-regulated target gene is stimulated by rtTA only in the presence of tetracycline or doxycycline.

Methods for assessing the induction of alpha-synuclein following administration of an inducer (e.g., tetracycline or doxycycline) include western blotting using an antibody specific for alpha-synuclein or RT-PCR or northern blotting techniques to detect mRNA expression of alpha-synuclein. Such methods are well known to those in the art and are described in detail in Sambrook et al. (in Molecular Cloning: A Laboratory Manual. Cold Spring Harbor Laboratory Press, NY, Vol. 1, 2, 3 (1989)). Expression of alpha-synuclein can be detected at about one hour (e.g., about 30 minutes, about 90 minutes, about two hours, about three hours, about 4 hours, about 6 hours, about 8 hours, about 12 hours, about 12 hours, about 16 hours, about 20 hours, about 24 hours, about 36 hours, about 48 hours or more) post-induction (see Example 1). Maximum induction levels are often observed about 12-24 hours post induction. In some embodiments, one can assess (evaluate) a change in expression over time post-induction. For example, the amount of expression of alpha-synuclein before induction (i.e., before the inducer (e.g., doxycycline or tamoxifen) is added) can be compared to the expression of alpha-synuclein by the cells at various time points (e.g., 1, 4, 8, and 12 hours; 4, 8, 12 and 16 hours; 8, 12, 16, and 20 hours; 12, 24, 36, and 48 hours) post-induction.

A suitable starting concentration of an inducing agent is about 0.001 μM (e.g., about 0.01 μM, about 0.1 μM, about 1.0 μM, about 10.0 μM, about 100 μM). It is understood that the concentration of the inducing agent can be optimized for the particular experiment and can depend on, for example, the cell line, the transgene, or the culture conditions the cells are grown in (e.g., low serum conditions).

In some embodiments, a mammalian cell (e.g., a mammalian neuronal cell) containing a nucleic acid vector encoding alpha-synuclein under control of an inducible promoter is in a non-human mammal (e.g., mouse, rat, or guinea pig). The vector can be introduced to the mammalian cell (e.g., a neuronal cell) ex vivo, i.e., the cell can be transfected in vitro and then implanted or otherwise delivered to the mammal (e.g., surgically implanted). Alternatively, a non-human transgenic animal can be established wherein the nucleic acid vector using any of a variety of techniques known in the art (see, for example, Manson et al. (2001) Exp. Rev. Mol. Med. 11; and Hofker et al. Trangenic Mouse: Methods and Protocols (Methods in Molecular Biology) Humana Press, Clifton, N.J., Vol. 29 (2002)).

Induction of expression of alpha-synuclein in an animal can be accomplished by administering to the animal an appropriate amount of the inducer. The inducer can be delivered to the mammal as part of food or water (i.e., in the food or water) or can be administered intravenously or parenterally (e.g., subcutaneous injection).

Suitable dosages of inducing agent and methods for detecting induction of a transgene in an animal model are described in, for example, Teng et al. (2002) Physiol. Genomics 11:99-107; Kim et al. (2003) Am. J. Pathol. 162(5):1693-1707; and Zabala et al. (2004) Cancer Res. 64:2799-2804.

It is understood that any of the in vitro or in vivo embodiments of the mammalian cell described above can be used in the following screening methods.

### Screening Methods

The invention features methods of screening for and identifying a “candidate agent” (e.g., a compound or a drug) that prevents or suppresses cytotoxicity resulting from overexpression of alpha-synuclein in a mammalian neuronal cell. Thus, a “candidate agent,” as referred to herein, is any substance with a potential to reduce, interfere with or curtail (i.e., prevent or suppress) cytotoxicity resulting from overexpression of alpha-synuclein in a mammalian neuronal cell. It should be understood that the cytotoxicity resulting from overexpression of alpha-synuclein in a mammalian neuronal cell can be, e.g., apoptosis or necrosis, and can be the result of, but not limited to, impaired proteasomal activity in the cell or the formation of inclusions/aggregation in the cytoplasm. However, irrespective of the exact mechanism of action, agents identified by the screening methods herein could be useful in treating synucleinopathies in a subject (e.g., a human patient) such as, but not limited to, Parkinson's disease, familial Parkinson's disease, Lewy body disease, the Lewy body variant of Alzheimer's disease, dementia with Lewy bodies, multiple system atrophy, or the Parkinsonism-dementia complex of Guam.

Various types of candidate agents can be screened by the methods described herein, including, but not limited to, nucleic acids, polypeptides, small molecule compounds, large molecule compounds, peptidomimetics or any other compounds described herein (e.g., see “Compounds” below). In some instances, the candidate agents are genetic agents that reduce, interfere with, or curtail cytotoxicity resulting from overexpression of alpha-synuclein in a mammalian neuronal cell. For example, a cDNA library containing coding sequences for a variety genes can be screened to identify potential therapeutic genes for the diseases described herein. Alternatively, a screen can be performed to identify genetic elements that contribute to cytotoxicity resulting from alpha-synuclein expression in a mammalian cell. For example, a library of siRNAs or antisense oligonucleotides can be screened such that the level or amount of cytotoxicity in the absence of one or more genes could be determined. In another example, a mammalian neuronal cell could be mutagenized to inactivate one or more genes prior to performing the screening assay. In these examples, a reduced level of alpha-synuclein-induced cytotoxicity in a cell in the absence of a gene (through mutational inactivation or silencing) indicates that the gene contributes to alpha-synuclein-induced cytotoxicity. Accordingly, siRNAs or antisense oligonucleotides that target that gene, for example, can be useful in treating a synucleinopathy.

Screening methods to identify an agent capable of preventing or suppressing cytotoxicity resulting from overexpression of an alpha-synuclein can involve the steps of: (i) culturing the cell in the presence of a candidate agent and under conditions that allow for expression of the nucleic acid encoding an alpha-synuclein at a level that, in the absence of the candidate agent, is sufficient to induce toxicity in the cell; (ii) measuring cell viability in the presence of the candidate agent; and (iii) comparing cell viability measured in the presence of the candidate agent to cell viability in the absence of the candidate agent, where if cell viability is greater in the presence of the candidate agent as compared to in the absence of the candidate agent, then the candidate agent is identified as a compound that prevents or suppresses alpha-synuclein-induced toxicity.

The screening assays can involve a mammalian cell containing a stably integrated nucleic acid encoding an alpha-synuclein under the control of an inducible promoter. Although the cell can be any mammalian cell, preferably the cell is a neuronal cell (e.g., primary neuronal cells or a neural cell line such as PC12, H4, SK-N-SH, SH-SY5Y, Neuro-2a, SVG p12, CCF-STTG1, SW 1088, SW 1783, LN-18, A172, U-138 MG, T98G, U-87 MG, U-118 MG, Hs 683, M059K, M059J, H4, LN-229, Daoy, or PFSK-1 (additional cell lines are available at the American Type Culture Collection (ATCC), Manassass, Va.). Alpha-synuclein can be under the control of a tetracycline-inducible promoter (as is described in the Examples) or any other suitable inducible promoter.

Cells containing a stably integrated alpha-synuclein under the control of an inducible promoter (e.g., a tetracycline-inducible promoter) can be grown in tissue culture plates, ideally, multi-well assay plates such as 96 well culture plates. Cells can be cultured in the absence (Uninduced) or presence (Induced) of an inducing agent (e.g., tetracycline or doxycycline) to induce expression of alpha-synuclein. Uninduced and induced cells can also be treated with a candidate compound (e.g., one dose of a candidate agent, e.g., a compound). Induced or uninduced cells cultured in the absence of a compound can optionally be treated with a like amount or concentration of the medium in which the candidate agent was delivered (e.g., DMSO). The assay can include cells or sets of cells as follows: (i) Induced cells treated with a candidate compound; (ii) Induced cells not treated with a candidate compound; (iii) Uninduced cells treated with a candidate compound; and (iv) Uninduced cells not treated with a candidate compound. To determine if a candidate compound prevents or suppresses alpha-synuclein-induced cytotoxicity in a mammalian cell, the amount of toxicity present in cell set (i) can be compared to the amount of cytotoxicity present in cell set (ii). If more cytotoxicity is present in cell set (ii) than is present in cell set (i), this is an indication that the agent prevents or suppresses alpha-synuclein-induced cytotoxicity in the cells. Cell sets (iii) and (iv) can be used as controls to, for example, normalize the amount of cytotoxicity in cells due to the culture conditions irrespective of inducing agent or compound respectively. For example, the amount of toxicity present in cell set (iii) can indicate if a candidate agent is itself cytotoxic.

The cells can be treated with two or more concentrations of a compound, where, for example, a concentration-dependence or EC50 is to be determined (see below). Suitable concentrations of a candidate compound for the assay include, for example, about 0.01 μM to 1 mM of the agent (e.g., about 0.01 μM to 0.1 μM, about 0.1 μM to 1 μM, about 1 μM to 10 μM, about 10 to 1 mM, or about 100 μM to 1 mM).

It is understood that some optimization can be required to determine a suitable amount of inducing agent or compound for the method. Such optimization can be based on, for example, the type of cell used, the specific compound, the amount of alpha-synuclein induction, or the time required before induction of alpha-synuclein.

Methods of assessing the efficacy of an agent to prevent or suppress alpha-synuclein-induced cytotoxicity can be quantitative, semi-quantitative, or qualitative. Thus, for example, the activity of an agent can be determined as a discrete value. An example of a quantitative determination of an agent's is a 50% Effective Concentration, or EC50 value, which is the molar concentration of an agent (e.g., a compound) that gives one-half the maximal response of that agent. Alternatively, the efficacy of an agent can be assessed using a variety of semi-quantitative/qualitative systems known in the art. Thus, the efficacy of an agent to prevent or suppress alpha-synuclein-induced cytotoxicity in a mammalian cell can be expressed as, for example, (a) one or more of “excellent”, “good”, “satisfactory”, “unsatisfactory”, and/or “poor”; (b) one or more of “very high”, “high”, “average”, “low”, and/or “very low”; or (c) one or more of “+++++”, “++++”, “+++”, “++”, “+”, “±”, and/or “−”.

Methods for determining the efficacy of agents in preventing or suppressing alpha-synuclein-induced cytotoxicity in a mammalian cell (e.g., a compound such as any of those described herein). Cells are generally plated on solid support matrix (e.g., a plastic tissue culture plate, or a multi-well (96 or 386-well) tissue culture plate) and grown in appropriate medium. Cells are then contacted with serial dilutions of a candidate agent generally ranging, for example, from 10 μM to 0.1 μM concentration. Often, a control compound (e.g., a known inhibitor of known concentration) is also added to a set of cells as an internal standard. Often, a set of cells are grown in the presence of a carrier, buffer, or solvent, in which the compound is delivered. Cells are grown in the presence or absence of test compounds for varying times, for example, from 1 to three days (1 day, 2 days, 3 days, 4 days, 1 week, 2 weeks), followed by a test for the number of cells remaining on the plate or the viability of the cells remaining on the plate. Methods of detecting (e.g., determining or measuring) the extent of alpha-synuclein-induced cytotoxicity in the presence or absence of an agent are myriad and well known to those of ordinary skill in the art. These methods can include, for example, measuring ATP concentration in a cell. The amount of ATP present in a cell or population of cells is proportional to the number of viable cells in that population. In one example, ATP concentration can be determined enzymatically, for example, by using luciferase/luciferin. These enzymes produce a light signal in a reaction requiring ATP hydrolysis. Thus, the more ATP present in a sample, the more light produced. In this method, cells are first harvested and lysed. Cell lysates are then incubated with luciferase/luciferin and the amount of ATP-dependent light produced from the sample can be detected and/or quantitated using a luminometer (e.g., Turner BioSystems TD-20/20 Luminometer, Turner Biosystems, Sunnyvale, Calif.). In this case, to determine the efficacy of a given agent in preventing or suppressing alpha-synuclein induced cytotoxicity, the amount of light signal produced from induced cells in the presence of the compound can be compared to the light signal produced from induced cells in the absence of the agent. Where more light signal is produced from lysates of cells cultured in the presence of the agent as compared to cells cultured in the absence of the agent, this indicates that the compound prevents or suppresses cytotoxicity. Further examples of this method are set forth in the Examples.

Other suitable methods for determining the efficacy of agents in preventing or suppressing alpha-synuclein-induced cytotoxicity include, for example, counting the number of cells remaining after the period of induction in the absence or presence of the agent. In this method, cells can be trypsinized from the plate, washed, stained with a dye (e.g., trypan blue), and counted using a microscope or mechanical cell counter (Beckman-Coulter Z1™ Series COULTER COUNTER® Cell and Particle Counter). Since dyes like trypan blue are only taken up by dead or dying cells, this method allows for discrimination (i.e., blue or white cell) between viable and non-viable cells in a population. Another method for determining prevention or suppressor of alpha-synuclein-induced cytotoxicity by an agent (e.g., any one of the compositions described herein) following treatment is a metabolic assay, for example, an MTT-metabolic assay (Invitrogen, USA). MTT Diphenyltetrazolium Bromide, is a tetrazolium salt (yellowish) that is cleaved to formazan crystals by the succinate dehydrogenase system which belongs to the mitochondrial respiratory chain, and is only active in viable cells. The mitochondrial succinate dehydrogenase reduces the MTT crystals into purple formazan in the presence of an electron coupling reagent. Following the treatment of the cells with a compound, the cells are exposed to the MTT reagent and the more viable cells are present in a well, the more formazan dye is produced. The amount of formazan dye can be measured, for example, using a spectrophotometer.

Other commonly used methods of testing for prevention or suppression of cytotoxicity in a cell (e.g., cytotoxicity resulting from overexpression of alpha-synuclein in a mammalian cell) by an agent (e.g., a compound or a composition described herein) include the monitoring of DNA synthesis in the cell. For example, induced cells grown in the presence or absence of an agent are also treated with a nucleotide analog that can incorporate into the DNA of the cell upon cell division. Examples of such nucleotide analogs include, for example, BrdU or ³H-Thymidine. In each case, the amount of label incorporated into the induced cells (grown in the presence and absence of a given agent) is quantitated, and the amount of label incorporation is directly proportional to the amount of cell growth in the population of cells. The amount of label incorporated in the induced cells in the presence and absence of an agent can be normalized to the amount of label incorporated into uninduced cells. More signal (i.e., more DNA synthesis) in an induced cell set treated with the agent as compared to induced cells not treated with the agent indicates that the agent prevents or suppresses alpha-synuclein-induced cytotoxicity.

Other suitable methods for assessing suppression or prevention of alpha-synuclein-induced cytotoxicity by an agent include the detection of apoptosis in a cell. Such methods of detecting or measuring apoptosis include, for example, monitoring DNA fragmentation, caspase activation, or annexin V expression.

The invention also features screening methods to identify a compound that prevents or suppresses alpha-synuclein-induced Golgi fragmentation. For example, induced cells cultured in the presence and absence of an agent can be: (i) fixed (e.g., with formaldehyde or paraformaldehyde); (ii) treated with a detectably-labeled first antibody specific for Golgi-specific protein marker; and (iii) the signal produced by the detectable label can be detected using any of a number of methods, including fluorescence-assisted cell sorting (FACS) and confocal microscopy. In one example, a first antibody to the membrane bound, Golgi-localized protein, GM130, can be used to specifically detect the metes and bounds of a Golgi. In some instances, a punctuate pattern is detected indicating an intact Golgi. A more diffuse Golgi staining generally indicates a disruption in the integrity of the organelle. Exemplary methods for determining Golgi integrity are set forth in the Examples and are also described, e.g., in Gosavi et al. (2002) J. Biol. Chem. 277(50):48984-48992.

The detectable label can be conjugated to the first antibody (the primary antibody which specifically recognizes the Golgi-specific protein markers) or on a secondary antibody which is capable of binding to the first antibody. Alternatively, the first antibody can be conjugated to the first member of a binding pair (i.e., streptavidin or biotin) and the second member of the binding pair can be linked to the detectable moiety. The detectable moiety can include radiolabels (e.g., ¹²⁵I, ³⁵S, ³³P, or ³²P), fluorescent labels (e.g., texas red, fluorescein), a luminescent moiety (e.g., a lanthanide), or a one or more members of a FRET pair. Methods of detecting these detectable markers are well known in the art and set forth in the Examples below.

A block in ER to Golgi vesicular trafficking has been observed following induction of alpha-synuclein expression (see PCT Publication No. WO 2006/073734 which is incorporated by reference in its entirety). Suitable assays for monitoring vesicular trafficking between the ER and Golgi generally involve monitoring the trafficking of specific proteins between these two organelles. For example, the proteins can be endogenous proteins destined for secretion and can be detected by, for example, any of the fixation and antibody-based staining methods described herein. Alternatively, the protein or proteins can be detectable proteins (e.g., a green fluorescent protein or a protein conjugated to a fluorescent protein) in which case the proteins can be directly visualized in the cell. In these assays, the subcellular localization of the proteins that traffic through the ER to Golgi pathway is monitored. As expression of alpha-synuclein blocks transport of proteins from the ER to the Golgi, candidate agents that promote or increase transport of proteins form the ER to the Golgi are thus identified as compounds that increase ER to Golgi vesicular transport. These compounds are expected to be candidate therapeutic agents for reducing alpha-synuclein mediated toxicity and treating synucleinopathies (e.g., any of the synucleinopathies described herein). Exemplary methods of detecting ER to Golgi trafficking for use in the methods described herein can be found in, e.g., Kawano et al. (2006) J. Biol. Chem. 281(40):30279-30288; Kumagai et al. (2005) J. Biol. Chem. 280(8):6488-6495; and Giussani et al. (2006) Mol. Cell. Biol. 26(13):5055-5069.

Screening methods can also be performed to identify compounds that, in alpha-synuclein expressing cells, modulate donor vesicle (e.g., ER vesicle or synaptic vesicle) fusion to acceptor membranes (e.g., Golgi membrane or presynaptic membrane). Compounds that increase donor vesicle fusion to acceptor membranes in alpha-synuclein expressing cells are expected to be candidate therapeutic agents for reducing alpha-synuclein mediated toxicity and treating synucleinopathies. Exemplary methods of detecting vesicle docking and fusion for use in the methods described herein can be found in, for example, Hibbert et al. (2006) Int. J. Biochem. Cell Biol. 38(3):461-471; Tsuboi et al. (2005) J. Biol. Chem. 280(47):39253-39259; and Huang et al. (2005) Mol. Biol. Cell 16(6):2614-2623.

The invention also discloses methods for identifying a compound that increases protein secretion from a cell. It is understood that such methods can also be used to assess alpha-synuclein induced cytotoxicity in a mammalian cell (e.g., a neuronal cell). For example, the method can involve (i) culturing induced cells in the presence and absence of a candidate agent; (ii) measuring the amount of one or more proteins secreted from the cell in the presence of a candidate agent; and (iii) comparing the amount of one or more proteins secreted from a cell in the presence of the candidate agent to the amount of one or more proteins secreted in the absence of the candidate agent. An elevated (increased) secretion of the one or more proteins in the presence of the candidate agent as compared to in the absence of the candidate agent indicates that candidate agent is a compound that can increase secretion of the one or more proteins. Suitable methods for detecting protein expression from a cell are well known in the art. For example, the medium from cultured cells can be collected and analyzed for the presence or amount of a protein by, e.g., western or dot-blotting or enzyme-linked immunosorbent assays (ELISA). Where the protein is secreted at low levels, the medium can optionally be concentrated. Alternatively, where the secreted protein to be detected is, for example, an enzyme, enzymatic activity in the cell culture medium can be detected or quantitated, e.g., through the use of a colorimetric substrate. In some instances, the secreted protein will remain bound to the surface of the cell and can be detected using, for example, FACS or confocal microscopy techniques. In some instances, the secreted protein can be detected in transit to the cell surface in situ (i.e., in the cell) by, e.g., fixation and antibody-based staining as described above or where the protein can be directly detected (e.g., a fluorescent protein) the protein can be detected or quantitated by FACS or microscopy techniques.

It should be understood that the screening methods described herein can be also be used as secondary, or cell-based screens to identify compounds useful in treating a synucleinopathy. For example, the screening methods can be used following a primary screen where, for example, a compound is first selected based on an ability to inhibit alpha-synuclein induced'toxicity in another system (e.g., yeast).

Exemplary compounds useful, for example, as positive controls in any of the screening methods described herein include 1-t-Butyl-3-(4-chloro-phenyl)-1H-pyrazolo[3,4-d]pyrimidin-4-ylamine and forskolin (see Example 5) and those found in, for example, PCT Publication No. WO 2006/034003, which is incorporated by reference in its entirety.

Screening assays can be performed in any format that allows for rapid preparation, processing, and analysis of multiple reactions. This can be, for example, in multi-well assay plates (e.g., 96 wells or 386 wells). Stock solutions for various agents can be made manually or robotically, and all subsequent pipetting, diluting, mixing, distribution, washing, incubating, sample readout, data collection and analysis can be done robotically using commercially available analysis software, robotics, and detection instrumentation capable of detecting the signal generated from the assay. Examples of such detectors include, but are not limited to, spectrophotometers, luminometers, fluorimeters, and devices that measure radioisotope decay.

### Compounds

Compounds to be screened or identified using any of the methods described herein can include various chemical classes, though typically small organic molecules having a molecular weight in the range of 50 to 2,500 daltons. These compounds can comprise functional groups necessary for structural interaction with proteins (e.g., hydrogen bonding), and typically include at least an amine, carbonyl, hydroxyl, or carboxyl group, and preferably at least two of the functional chemical groups. These compounds often comprise cyclical carbon or heterocyclic structures and/or aromatic or polyaromatic structures (e.g., purine core) substituted with one or more of the above functional groups.

In alternative embodiments, compounds can also include biomolecules including, but not limited to, peptides, polypeptides, peptidomimetics (e.g., peptoids), amino acids, amino acid analogs, saccharides, fatty acids, steroids, purines, pyrimidines, derivatives or structural analogues thereof, polynucleotides, nucleic acid aptamers, and polynucleotide analogs.

Compounds can be identified from a number of potential sources, including: chemical libraries, natural product libraries, and combinatorial libraries comprised of random peptides, oligonucleotides, or organic molecules. Chemical libraries consist of diverse chemical structures, some of which are analogs of known compounds or analogs or compounds that have been identified as “hits” or “leads” in other drug discovery screens, while others are derived from natural products, and still others arise from non-directed synthetic organic chemistry. Natural product libraries re collections of microorganisms, animals, plants, or marine organisms which are used to create mixtures for screening by: (1) fermentation and extraction of broths from soil, plant or marine microorganisms, or (2) extraction of plants or marine organisms. Natural product libraries include polypeptides, non-ribosomal peptides, and variants (non-naturally occurring) thereof. For a review, see Science 282:63-68 (1998). Combinatorial libraries are composed or large numbers of peptides, oligonucleotides, or organic compounds as a mixture. These libraries are relatively easy to prepare by traditional automated synthesis methods, PCR, cloning, or proprietary synthetic methods. Of particular interest are non-peptide combinatorial libraries. Still other libraries of interest include peptide, protein, peptidomimetic, multiparallel synthetic collection, recombinatorial, and polypeptide libraries. For a review of combinatorial chemistry and libraries created therefrom, see Myers, Curr. Opin. Biotechnol. 8:701-707 (1997). Identification of test compounds through the use of the various libraries herein permits subsequent modification of the test compound “hit” or “lead” to optimize the capacity of the “hit” or “lead” to prevent or suppress alpha-synuclein-induced cytotoxicity in a mammalian cell.

The compounds identified above can be synthesized by any chemical or biological method. The compounds identified above can also be pure, or may be in a heterologous composition (e.g., a pharmaceutical composition), and can be prepared in an assay-, physiologic-, or pharmaceutically-acceptable diluent or carrier (see Pharmaceutical Compositions and Methods of Treatment below).

### Pharmaceutical Compositions

An agent found to prevent or suppress alpha-synuclein-induced cytotoxicity in a mammalian neuronal cell can be formulated as a pharmaceutical composition, e.g., for administration to a subject to treat a synucleinopathy, such as Parkinson's disease, familial Parkinson's disease, Lewy body disease, the Lewy body variant of Alzheimer's disease, dementia with Lewy bodies, multiple system atrophy, or the Parkinsonism-dementia complex of Guam. Typically, a pharmaceutical composition includes a pharmaceutically acceptable carrier. As used herein, “pharmaceutically acceptable carrier” includes any and all solvents, dispersion media, coatings, antibacterial and antifungal agents, isotonic and absorption delaying agents, and the like that are physiologically compatible. The composition can include a pharmaceutically acceptable salt, e.g., an acid addition salt or a base addition salt (see e.g., Berge et al., J. Pharm. Sci. 66:1-19, 1977).

The agent can be formulated according to standard methods. Pharmaceutical formulation is a well-established art, and is further described, e.g., in Gennaro (ed.), Remington: The Science and Practice of Pharmacy, 20th ed., Lippincott, Williams & Wilkins (2000) (ISBN: 0683306472); Ansel et al., Pharmaceutical Dosage Forms and Drug Delivery Systems, 7th Ed., Lippincott Williams & Wilkins Publishers (1999) (ISBN: 0683305727); and Kibbe (ed.), Handbook of Pharmaceutical Excipients American Pharmaceutical Association, 3rd ed. (2000) (ISBN: 091733096X).

In one embodiment, an agent that prevents or suppresses alpha-synuclein-induced cytotoxicity in a mammalian cell can be formulated with excipient materials, such as sodium chloride, sodium dibasic phosphate heptahydrate, sodium monobasic phosphate, and a stabilizer. It can be provided, for example, in a buffered solution at a suitable concentration and can be stored at 2-8° C.

The pharmaceutical compositions may be in a variety of forms. These include, for example, liquid, semi-solid and solid dosage forms, such as liquid solutions (e.g., injectable and infusible solutions), dispersions or suspensions, tablets, pills, powders, liposomes and suppositories. The preferred form can depend on the intended mode of administration and therapeutic application. Typically compositions for the agents described herein are in the form of injectable or infusible solutions.

Such compositions can be administered by a parenteral mode (e.g., intravenous, subcutaneous, intraperitoneal, or intramuscular injection). The phrases “parenteral administration” and “administered parenterally” as used herein mean modes of administration other than enteral and topical administration, usually by injection, and include, without limitation, intravenous, intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal, epidural, intracerebral, intracranial, intracarotid and intrasternal injection and infusion. The composition can be formulated as a solution, microemulsion, dispersion, liposome, or other ordered structure suitable for stable storage at high concentration. Sterile injectable solutions can be prepared by incorporating an agent described herein in the required amount in an appropriate solvent with one or a combination of ingredients enumerated above, as required, followed by filtered sterilization. Generally, dispersions are prepared by incorporating an agent described herein into a sterile vehicle that contains a basic dispersion medium and the required other ingredients from those enumerated above. In the case of sterile powders for the preparation of sterile injectable solutions, the preferred methods of preparation are vacuum drying and freeze-drying that yields a powder of an agent described herein plus any additional desired ingredient from a previously sterile-filtered solution thereof. The proper fluidity of a solution can be maintained, for example, by the use of a coating such as lecithin, by the maintenance of the required particle size in the case of dispersion and by the use of surfactants. Prolonged absorption of injectable compositions can be brought about by including in the composition an agent that delays absorption, for example, monostearate salts and gelatin.

In certain embodiments, the agent can be prepared with a carrier that will protect the compound against rapid release, such as a controlled release formulation, including implants, and microencapsulated delivery systems. Biodegradable, biocompatible polymers can be used, such as ethylene vinyl acetate, polyanhydrides, polyglycolic acid, collagen, polyorthoesters, and polylactic acid. Many methods for the preparation of such formulations are patented or generally known. See, e.g., Sustained and Controlled Release Drug Delivery Systems, J. R. Robinson, ed., Marcel Dekker, Inc., New York, 1978.

An agent identified as one that prevents or suppresses alpha-synuclein-induced cytotoxicity in a mammalian cell can be modified, e.g., with a moiety that improves its stabilization and/or retention in circulation, e.g., in blood, serum, or other tissues, e.g., by at least 1.5, 2, 5, 10, or 50 fold. The modified agent can be evaluated to assess whether it can reach treatment sites of interest (e.g., locations of Lewy bodies) such as can occur in synucleinopathies, such as Parkinson's disease (e.g., by using a labeled form of the agent).

For example, the agent can be associated with a polymer, e.g., a substantially non-antigenic polymer, such as a polyalkylene oxide or a polyethylene oxide. Suitable polymers will vary substantially by weight. Polymers having molecular number average weights ranging from about 200 to about 35,000 Daltons (or about 1,000 to about 15,000, and 2,000 to about 12,500) can be used. For example, a agent can be conjugated to a water soluble polymer, e.g., a hydrophilic polyvinyl polymer, e.g., polyvinylalcohol or polyvinylpyrrolidone. A non-limiting list of such polymers include polyalkylene oxide homopolymers such as polyethylene glycol (PEG) or polypropylene glycols, polyoxyethylenated polyols, copolymers thereof and block copolymers thereof, provided that the water solubility of the block copolymers is maintained. Additional useful polymers include polyoxyalkylenes such as polyoxyethylene, polyoxypropylene, and block copolymers of polyoxyethylene and polyoxypropylene (Pluronics); polymethacrylates; carbomers; and branched or unbranched polysaccharides. When the agent (e.g., a compound) is used in combination with a second agent (e.g., any additional therapies for synucleinopathies such as acetylcholinesterase inhibitors), the two agents can be formulated separately or together. For example, the respective pharmaceutical compositions can be mixed, e.g., just prior to administration, and administered together or can be administered separately, e.g., at the same or different times.

### Administration

A agent that can prevent or suppress alpha-synuclein-induced cytotoxicity in a mammalian cell can be administered to a subject, e.g., a human subject, by a variety of methods. For many applications, the route of administration is one of: intravenous injection or infusion (IV), subcutaneous injection (SC), intraperitoneally (IP), or intramuscular injection. In some cases, administration may be directly into the CNS, e.g., intrathecal, intracerebroventricular (ICV), intracerebral or intracranial. The agent can be administered as a fixed dose, or in a mg/kg dose. In other instances, administration can be oral (e.g., inhalation), transdermal (topical), transmucosal, or rectal.

Oral compositions generally include an inert diluent or an edible carrier. For the purpose of oral therapeutic administration, the active compound can be incorporated with excipients and used in the form of tablets, troches, or capsules, e.g., gelatin capsules. Oral compositions can also be prepared using a fluid carrier for use as a mouthwash. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring.

The powders and tablets contain from 1% to 95% (w/w) of the active compound. In certain embodiments, the active compound ranges from 5% to 70% (w/w). Suitable carriers are magnesium carbonate, magnesium stearate, talc, sugar, lactose, pectin, dextrin, starch, gelatin, tragacanth, methylcellulose, sodium carboxymethylcellulose, a low melting wax, cocoa butter, and the like. The term “preparation” is intended to include the formulation of the active compound with encapsulating material as a carrier providing a capsule in which the active component with or without other carriers, is surrounded by a carrier, which is thus in association with it. Similarly, cachets and lozenges are included. Tablets, powders, capsules, pills, cachets, and lozenges can be used as solid dosage forms suitable for oral administration.

Aqueous solutions suitable for oral use can be prepared by dissolving the active component in water and adding suitable colorants, flavors, stabilizers, and thickening agents as desired. Aqueous suspensions suitable for oral use can be made by dispersing the finely divided active component in water with viscous material, such as natural or synthetic gums, resins, methylcellulose, sodium carboxymethylcellulose, and other well-known suspending agents.

For administration by inhalation, the compounds are delivered in the form of an aerosol spray from pressured container or dispenser which contains a suitable propellant, e.g., a gas such as carbon dioxide, or a nebulizer.

Systemic administration can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the active compounds are formulated into ointments, salves, gels, or creams as generally known in the art.

The compounds can also be prepared in the form of suppositories (e.g., with conventional suppository bases such as cocoa butter and other glycerides) or retention enemas for rectal delivery.

Where the agent is a polypeptide or otherwise particularly antigenic, the dose can also be chosen to reduce or avoid production of antibodies against the agent. The route and/or mode of administration of the agent can also be tailored for the individual case.

Dosage regimens are adjusted to provide the desired response, e.g., a therapeutic response or a combinatorial therapeutic effect. The dosage regimen will, for example, prevent or suppress alpha-synuclein-induced cytotoxicity in one or more affected cells in a mammal having a synucleinopathy. Generally, a dose of an agent (e.g., a compound) is optionally formulated separately or together with an appropriate dose of a second therapeutic agent can be used to provide a subject with the agent. Suitable dosages and/or dose ranges for the agent include an amount sufficient to prevent or suppress alpha-synuclein-induced cytotoxicity in a subject.

A dose of an agent required to prevent or suppress alpha-synuclein-induced cytotoxicity can depend on a variety of factors including, for example, the age, sex, and weight of a subject to be treated. Other factors affecting the dose administered to the subject include, e.g., the type or severity of the synucleinopathy. For example, a patient with advanced Alzheimer's disease may require a administration of a different dosage of an agent that prevents or suppresses alpha-synuclein-induced cytotoxicity than a patient with a milder form of Alzheimer's disease. Other factors can include, e.g., other disorders concurrently or previously affecting the patient, the general health of the patient, the genetic disposition of the patient, diet, time of administration, rate of excretion, drug combination, and any other additional therapeutics that are administered to the patient. It should also be understood that a specific dosage and treatment regimen for any particular patient will depend upon the judgment of the treating physician. The amount of active ingredients will also depend upon the particular described compound and the presence or absence and the nature of the additional therapeutic agents in the composition.

Dosage unit form or “fixed dose” as used herein refers to physically discrete units suited as unitary dosages for the subjects to be treated; each unit contains a predetermined quantity of active compound calculated to produce the desired therapeutic effect (e.g., the prevention or suppression alpha-synuclein-induced cytotoxicity) in association with the required pharmaceutical carrier and optionally in association with the other agent. Suitable administration frequencies are described elsewhere herein.

A pharmaceutical composition can include a therapeutically effective amount of a agent found to prevent or suppress alpha-synuclein-induced cytotoxicity described herein. Such effective amounts can be determined based on the effect of the administered agent, or the combinatorial effect of an agent and secondary agent if more than one agent is used. A therapeutically effective amount of an agent can also vary according to factors such as the disease state, age, sex, and weight of the individual, and the ability of the compound to elicit a desired response in the individual, e.g., amelioration of at least one disorder parameter, e.g., amelioration of at least one symptom of a synucleinopathy, e.g., impaired or failing memory. A therapeutically effective amount is also one in which any toxic or detrimental effects of the composition is outweighed by the therapeutically beneficial effects.

The following are examples of the practice of the invention. They are not to be construed as limiting the scope of the invention in any way.

### Examples

### Example 1

### Conditional Overexpression of Alpha-Synuclein

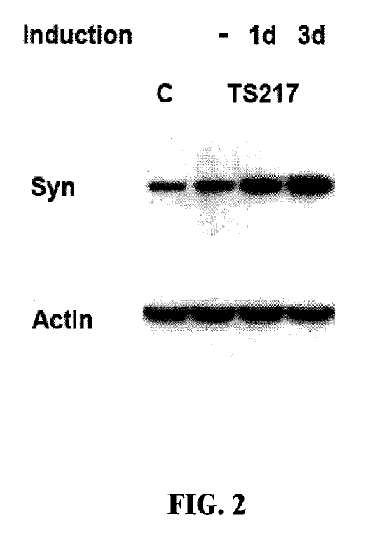

The TS217 cell line was generated from H4 human glioma cells stably expressing alpha-synuclein polypeptide under the control of tetracycline-on promoter (FIG. 1). To test for the induction of wild-type alpha-synuclein expression, the TS217 cells were treated without (“−”) or with 0.1 μg/mL tetracycline for 1 day (1d) and 3 days (3d) (see FIG. 2). Whole-cell lysates were prepared from the treated or untreated TS217 cells at the various times and cellular proteins were resolved by sodium-dodecyl-sulfate polyacrylamide gel electrophoresis (SDS-PAGE). Whole cell lysate from Parental H4 (C) was also prepared and subjected to SDS-PAGE to indicate endogenous level of alpha-synuclein protein. Expression of alpha-synuclein was confirmed by western blotting using an antibody specific for alpha-synuclein protein (FIG. 2). Expression of alpha-synuclein increased at least two-fold by 1 day post-induction and more than 3 or 4 fold by 3 days post-induction. These results indicated that the expression of alpha-synuclein was regulated by tetracycline.

### Example 2

### Cytotoxicity in Cells Overexpressing Alpha-Synuclein

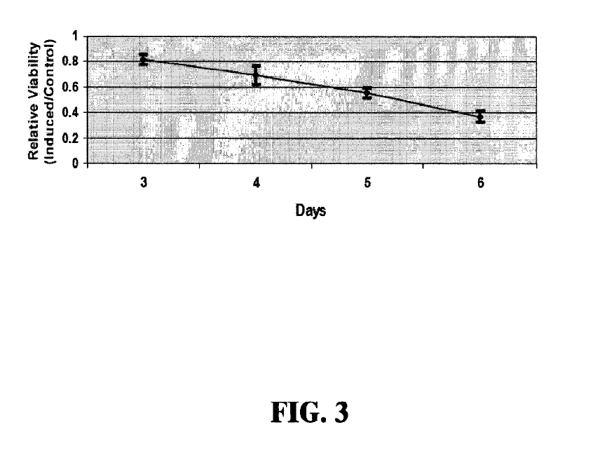

In some experimental systems, overexpression of alpha-synuclein has been shown to sensitize cells towards cell death induced by toxicity-inducing agents or conditions such as serum deprivation, dopamine, and low doses of proteasome inhibitors such as lactacystin (Smith et al. (2005) Hum. Mol. Genet. 14(24):3801-3811; Tabrizi et al. (2000) Hum. Mol. Genet. 9(18):2683-2689; and Ostreova et al. (1999) J. Neurosci. 19(14):5782-5791). To determine the effect of overexpression of wild-type alpha-synuclein on cell viability using the stable, inducible system, TS217 cells were treated with 0.1 μg/mL tetracycline for three to six days to induce expression of alpha-synuclein. The relative viability of the cells was assessed at one day intervals (day 3, day 4, day 5, and day 6) by measuring the cellular ATP level in cell lysates using a ViaLight® Plus Bioassay kit (Cambrex, Rockland, Me.). Relative cell viability was calculated as the ratio of induced cells to control cells (cells not treated with tetracycline), as an indication of alpha-synuclein-induced cytotoxicity (see FIG. 3). Relative cell viability decreased by over 50% from day 3 to day 6 in the absence of any other toxicity-inducing agents, indicating that expression of alpha-synuclein alone in these cells was capable of causing cell death.

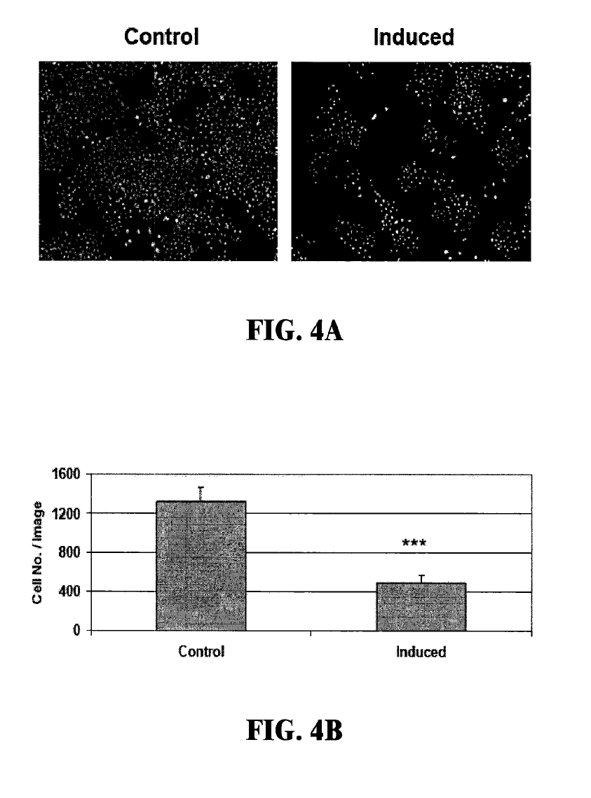

The cytotoxicity of alpha-synuclein overexpression in TS217 cells was also assessed using the membrane-permeable dye, calcein AM. TS217 cells were plated on glass tissue culture chamber slides coated with poly-L-lysine and cultured in the absence (Control) or presence (Induced) of 0.1 μg/mL tetracycline for 5 days to induce alpha-synuclein expression. Cells were treated with 2 μM calcein AM and the fluorescence of enzyme-activated calcein AM (in live cells) was visualized using a fluorescent microscope (Olympus, Center Valley, Pa.) fitted with a digital camera controlled by the Metamorph program (Universal Imaging, West Chester, Pa.). Calcein-positive cells were quantitated using Metamorph software (Universal Imaging, West Chester, Pa.) (FIG. 4A). Following the five day treatment with tetracycline (and induction of alpha-synuclein), the number of viable cells decreased by approximately 60% (FIG. 4B), further indicating that overexpression of alpha-synuclein in these cells results in cell death.

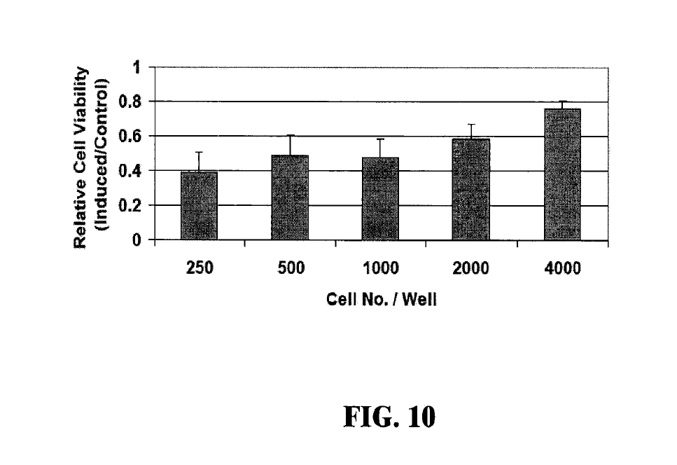

The effect of plating density on alpha-synuclein-induced toxicity was also investigated. TS217 cells were plated in 96 well plates at dilutions of 250, 500, 1000, 2000, and 4000 cells/well. Cells were incubated in the presence of tetracycline for five days and then harvested and lysed (as above) to measure intracellular ATP concentrations. More cell death was observed at lower cell density (e.g., 250 cells/well) than at higher cell density (e.g., 4000 cells/well), indicating that plating density has an effect on alpha-synuclein-induced toxicity (FIG. 10).

### Example 3

### Dose-Dependence of Tetracycline on Cell Viability

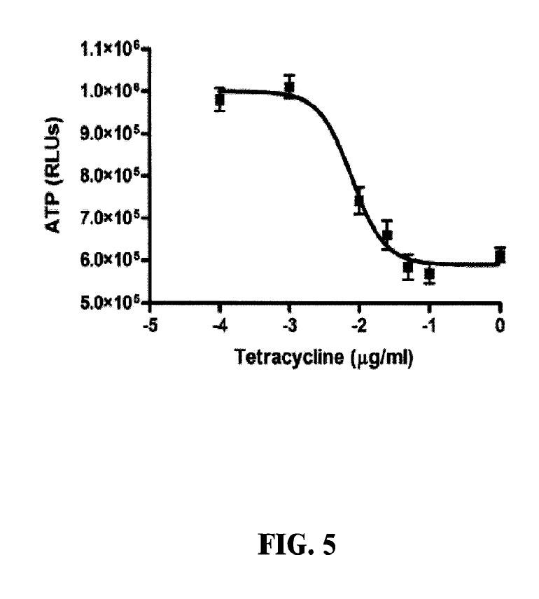

To determine whether the effect of tetracycline on cell viability was concentration-dependent, TS217 cells grown in 96 well plates were incubated with different concentrations of tetracycline for six days. Following treatment, each of the experimental cell groups were lysed and the cellular ATP measured as described above. Media with the vehicle alone (no tetracycline) served as control (FIG. 5). Relative cell viability, as a function of ATP concentration in the cell, decreased with increasing concentrations of tetracycline in a dose-dependent manner (see FIG. 5).

### Example 4

### Fragmentation of the Golgi Apparatus in Alpha-Synuclein

### Over-Expressing Cells

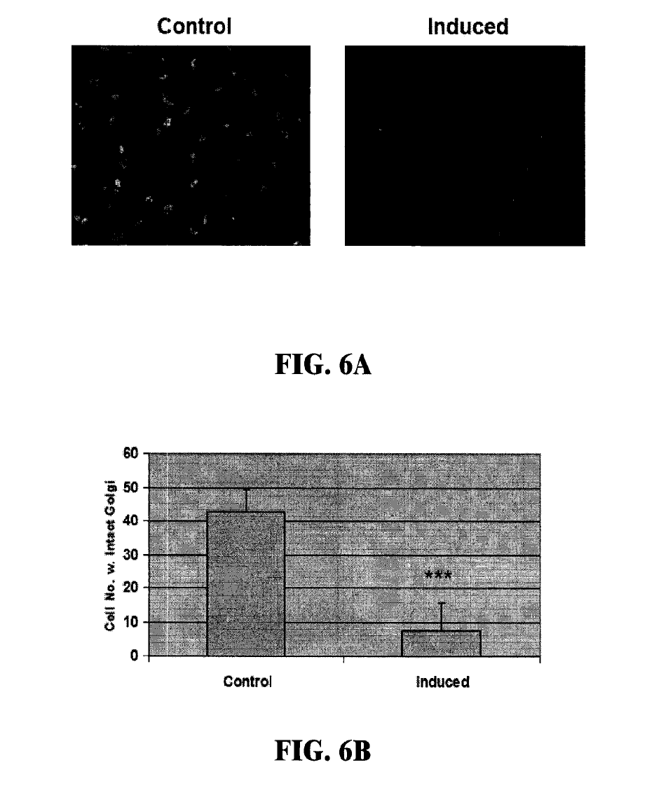

TS217 cells were plated on glass tissue culture chamber slides coated with poly-L-lysine and incubated for six days in the absence (Control) or presence of 0.1 μg/mL tetracycline (Induced) to induce alpha-synuclein expression. Following treatment, cells were fixed with 4% paraformaldehyde supplemented with 4% sucrose in phosphate-buffered saline, permeabilized with 0.2% triton in PBS, and stained with antibodies specific for the cis-Golgi tethering protein GM130 (FIG. 6A). The number of cells containing intact Golgi was reduced by almost 80% following the six day induction of alpha-synuclein (FIG. 6B).

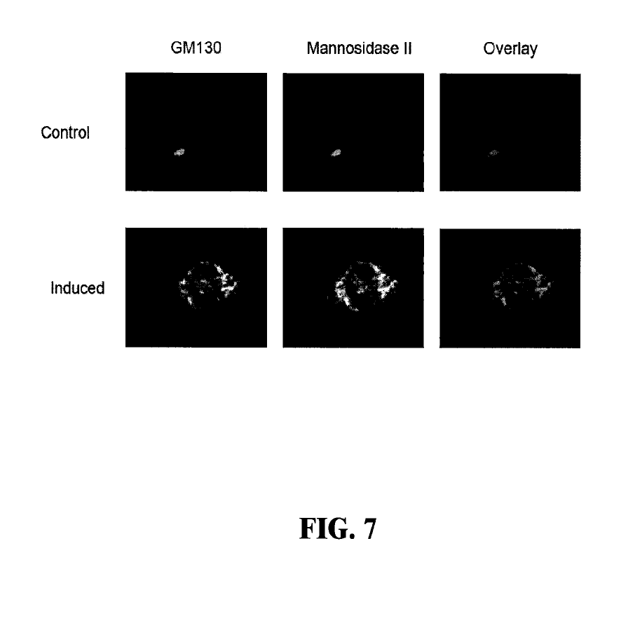

To further assess the effect of enforced overexpression of alpha-synuclein on Golgi integrity, TS217 cells treated for six days in the absence (Control) and presence (Induced) of tetracycline (see above) were double stained with antibodies specific for GM130 and the membrane-bound Golgi enzyme mannosidase II respectively. While Control cells exhibited a punctuate co-localized pattern for both GM130 and mannosidase II, Induced cells had a diffuse, non-localized Golgi pattern, further indicating that alpha-synuclein overexpression caused Golgi fragmentation in these cells (FIG. 7).

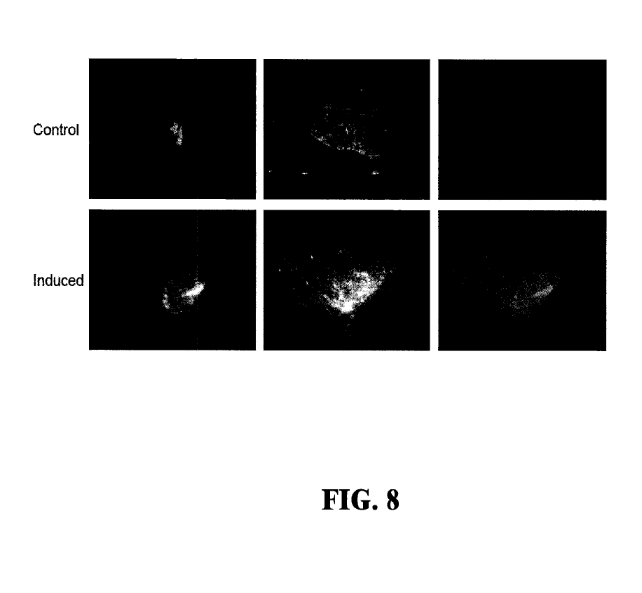

To determine if alpha-synuclein had a similar disruptive effect on endoplasmic reticulum (ER), TS217 cells were plated on glass tissue culture slides as before and cultured for five days in the absence (Control) and presence (Induced) of 0.1 μg/mL tetracycline to induce alpha-synuclein expression. Cells were fixed and stained with antibodies to the ER-localized Ca2+ binding protein Calnexin or mannosidase II as a control (FIG. 8). Although the Golgi apparatus displayed profound fragmentation (also see FIG. 7), no gross change in ER morphology was observed in cells overexpressing alpha-synuclein (FIG. 8).

### Example 5

### Dose-Dependent Rescue of Cell Viability

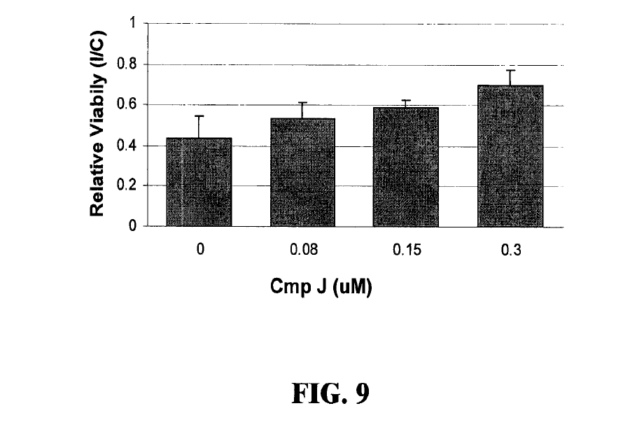

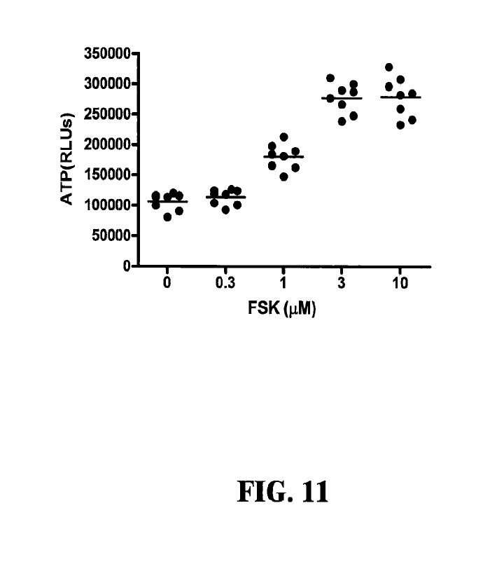

To determine if a select compound could inhibit alpha-synuclein-induced cytotoxicity, TS217 cells plated in 96 well tissue culture plates were cultured with 0.1 μg/mL tetracycline for 5 days in the presence of either 1-t-Butyl-3-(4-chloro-phenyl)-1H-pyrazolo[3,4-d]pyrimidin-4-ylamine (Compound J(Cmp J)) (0.08 μM, 0.15 μM, and 0.3 μM) or DMSO as a control (FIG. 9) or in the presence of either forskolin (0.3 μM, 1 μM, 3 μM, and 10 μM) or DMSO as a control (FIG. 11). After the five day treatment, cells were lysed and assayed for intracellular ATP concentration (as above) as a function of cell viability.

### Other Embodiments

It is to be understood that, while the invention has been described in conjunction with the detailed description thereof, the foregoing description is intended to illustrate and not limit the scope of the invention. Other aspects, advantages, and modifications of the invention are within the scope of the claims set forth below.

1. A mammalian neuronal cell comprising a stably integrated expression construct comprising an inducible promoter operably linked to a nucleic acid encoding a protein comprising a human alpha synuclein, wherein induction of expression of the nucleic acid, in the absence of co-treatment of the cell with a toxicity-inducing agent or a neuronal differentiation factor, results in a decrease in cell viability.

2. The cell of claim 1, wherein the alpha synuclein is wild type alpha-synuclein.

3. The cell of claim 1, wherein the alpha synuclein is a mutant alpha-synuclein.

4. The cell of claim 3, wherein the mutant alpha-synuclein is mutant human alpha-synuclein A53T.

5. The cell of claim 3, wherein the mutant alpha-synuclein is mutant human alpha-synuclein A30P.

6. The cell of claim 3, wherein the mutant alpha-synuclein is mutant human alpha-synuclein E46K.

7. The cell of claim 1, wherein the alpha synuclein is a full length alpha-synuclein.

8. The cell of claim 1, wherein the neuronal cell is a human neuronal cell.

9. The cell of claim 1, wherein the neuronal cell is derived from a neuronal cell line.

10. The cell of claim 1, wherein the neuronal cell is derived from the H4 cell line.

11. The cell of claim 1, wherein the neuronal cell is derived from the PC 12 cell line

12. The cell of claim 1, wherein the protein is a fusion protein comprising a detectable protein.

13. The cell of claim 12, wherein the detectable protein is a fluorescent protein, an enzyme, or an epitope.

14. The cell of claim 13, wherein the detectable protein is a fluorescent protein selected from the group consisting of a red fluorescent protein, green fluorescent protein, blue fluorescent protein, yellow fluorescent protein, and cyan fluorescent protein.

15. The cell of claim 1, wherein the cell is an isolated cell.

16. The cell of claim 1, wherein the cell further comprises a stably integrated repressor construct that constitutively expresses a repressor protein, wherein (i) in the absence of an exogenously added inducer, the repressor protein binds to the inducible promoter and suppresses expression of the nucleic acid, and (ii) in the presence of the exogenously added inducer, the repressor protein binds to the inducer and the nucleic acid is expressed.

17. The cell of claim 16, wherein the repressor protein is a Tet repressor

18. The cell of claim 16, wherein the inducer is tetracycline or doxycycline.

19. A non-human mammal comprising the neuronal cell of claim 1.

20. A method of inducing toxicity in a mammalian neuronal cell, the method comprising inducing a level of expression of the nucleic acid in the cell of claim 1 that is toxic to the cell.

21. A method of inducing toxicity in a mammalian neuronal cell, the method comprising contacting the cell of claim 16 with an amount of the exogenously added inducer effective to induce expression of the nucleic acid and toxicity in the cell.

22. A method of identifying a compound that prevents or suppresses alpha-synuclein-induced toxicity, the method comprising:
culturing the cell of claim 1 in the presence of a candidate agent and under conditions that allow for expression of the nucleic acid at a level that, in the absence of the candidate agent, is sufficient to induce toxicity in the cell;
measuring cell viability in the presence of the candidate agent; and
comparing cell viability measured in the presence of the candidate agent to cell viability in the absence of the candidate agent,
wherein if cell viability is greater in the presence of the candidate agent as compared to in the absence of the candidate agent, then the candidate agent is identified as a compound that prevents or suppresses alpha-synuclein-induced toxicity.

23. A method of identifying a compound that prevents or suppresses alpha-synuclein-induced toxicity, the method comprising:
culturing the cell of claim 16 in the presence of a candidate agent and an amount of the exogenously added inducer effective to induce expression of the nucleic acid at a level that, in the absence of the candidate agent, induces toxicity in the cell;
measuring cell viability in the presence of the candidate agent; and
comparing cell viability measured in the presence of the candidate agent to cell viability in the absence of the candidate agent,
wherein if cell viability is greater in the presence of the candidate agent as compared to in the absence of the candidate agent, then the candidate agent is identified as a compound that prevents or suppresses alpha-synuclein-induced toxicity.

24. A method of identifying a compound that prevents or suppresses alpha-synuclein-induced Golgi fragmentation, the method comprising:
culturing the cell of claim 16 in the presence of a candidate agent and an amount of the exogenously added inducer effective to induce expression of the nucleic acid at a level that, in the absence of the candidate agent, induces Golgi-fragmentation in the cell;
measuring Golgi fragmentation in the cell in the presence of the candidate agent; and
comparing Golgi fragmentation in the cell measured in the presence of the candidate agent to Golgi fragmentation in the absence of the candidate agent,
wherein if Golgi fragmentation is less in the presence of the candidate agent as compared to in the absence of the candidate agent, then the candidate agent is identified as a compound that prevents or suppresses alpha-synuclein-induced Golgi fragmentation.

25. A method of identifying a compound that increases endoplasmic reticulum to Golgi vesicular trafficking, the method comprising:
culturing the cell of claim 16 in the presence of a candidate agent and an amount of the exogenously added inducer effective to induce expression of the nucleic acid at a level that, in the absence of the candidate agent, reduces endoplasmic reticulum to Golgi vesicular trafficking in the cell;
measuring endoplasmic reticulum to Golgi vesicular trafficking in the cell in the presence of the candidate agent; and
comparing endoplasmic reticulum to Golgi vesicular trafficking in the cell measured in the presence of the candidate agent to endoplasmic reticulum to Golgi vesicular trafficking measured in the absence of the candidate agent,
wherein an increase in endoplasmic reticulum to Golgi vesicular trafficking in the cell in the presence of the candidate agent as compared to endoplasmic reticulum to Golgi vesicular trafficking in the absence of the candidate agent identifies the candidate agent as a compound that increases endoplasmic reticulum to Golgi vesicular trafficking

26. A method of identifying a compound that increases vesicle docking and fusion, the method comprising:
culturing the cell of claim 16 in the presence of a candidate agent and an amount of the exogenously added inducer effective to induce expression of the nucleic acid at a level that, in the absence of the candidate agent, reduces vesicle docking and fusion in the cell;
measuring vesicle docking and fusion in the cell in the presence of the candidate agent; and
comparing vesicle docking and fusion in the cell in the presence of the candidate agent to vesicle docking and fusion in the absence of the candidate agent,
wherein an increase in vesicle docking and fusion in the presence of the candidate agent as compared to vesicle docking and fusion in the absence of the candidate agent identifies the candidate agent as a compound that increases vesicle docking and fusion.

27. A method of identifying a compound that increases the secretion of a protein from a cell, the method comprising:
culturing the cell of claim 16 in the presence of a candidate agent and an amount of the exogenously added inducer effective to induce expression of the nucleic acid at a level that, in the absence of the candidate agent, reduces protein secretion from the cell;
measuring protein secretion from the cell in the presence of the candidate agent; and
comparing protein secretion from the cell in the presence of the candidate agent to protein secretion in the absence of the candidate agent,
wherein an increase in protein secretion from the cell in the presence of the candidate agent as compared to protein secretion in the absence of the candidate agent identifies the candidate agent as a compound that increases protein secretion from the cell.

28. A method of treating a synucleinopathy, the method comprising administering to an individual having, or at risk of developing, a synucleinopathy a pharmaceutical composition comprising a therapeutically effective amount of a compound identified by the method of claim 22.

29. The method of claim 28, wherein the synucleinopathy is Parkinson's disease, familial Parkinson's disease, Lewy body disease, the Lewy body variant of Alzheimer's disease, dementia with Lewy bodies, multiple system atrophy, or the Parkinsonism-dementia complex of Guam.


SEQUENCE LISTING (2 sequences)

SEQ ID NO: 1 - DNA, 423 bp, Homo sapiens
atggatgtattcatgaaaggactttcaaaggccaaggagggagttgtggctgctgctgagaaaaccaaacagggtgtggcagaagcagcaggaaagacaaaagagggtgttctctatgtaggctccaaaaccaaggagggagtggtgcatggtgtggcaacagtggctgagaagaccaaagagcaagtgacaaatgttggaggagcagtggtgacgggtgtgacagcagtagcccagaagacagtggagggagcagggagcattgcagcagccactggctttgtcaaaaaggaccagttgggcaagaatgaagaaggagccccacaggaaggaattctggaagatatgcctgtggatcctgacaatgaggcttatgaaatgccttctgaggaagggtatcaagactacgaacctgaagcctaa

SEQ ID NO: 2 - protein, 140 aa, Homo sapiens
MDVFMKGLSKAKEGVVAAAEKTKQGVAEAAGKTKEGVLYVGSKTKEGVVHGVATVAEKTKEQVTNVGGAVVTGVTAVAQKTVEGAGSIAAATGFVKKDQLGKNEEGAPQEGILEDMPVDPDNEAYEMPSEEGYQDYEPEA